(19) United States
(12) Patent Application Publication
(10) Pub. No.: US 2026/0315119 A1
(43) Pub. Date: Oct. 15, 2026

(54) System, Method And Device For Stabilizing A Distal Fracture To A Long Bone

(71) Applicant: Nikolaj Wolfson, San Francisco, CA (US)

(72) Inventor: Nikolaj Wolfson, San Francisco, CA (US)

(21) Appl. No.: 19/704,814

(22) Filed: Jun. 11, 2026

Related U.S. Application Data
(63) Continuation of application No. PCT/US24/60728, filed on Dec. 18, 2024.
(60) Provisional application No. 63/612,817, filed on Dec. 20, 2023.

(51) Int. Cl. A61B 17/72; A61B 17/00; A61B 17/56; A61B 17/68; A61B 17/80

(52) U.S. Cl. CPC A61B 17/7233; A61B 17/8061; A61B 2017/00477; A61B 2017/564; A61B 2017/681

(57) ABSTRACT

Systems and methods for stabilizing a bone having a distal end fracture and a distal fragment formed by the distal end fracture and a distal end of the bone, including an intramedullary nail, a plate, and a locking fastener. The intramedullary nail is configured to be coupled to the bone such that the intramedullary nail spans the distal end fracture between the bone and the distal fragment. The plate is configured to engage the distal fragment. The locking fastener is configured to couple the intramedullary nail and the plate in a first configuration in which the plate can rotate relative to the intramedullary nail and a second configuration in which the intramedullary nail and the plate are fixedly coupled together.

### CROSS-REFERENCE TO RELATED APPLICATIONS

This application is a continuation of PCT/US2024/060728, filed on 2024 Dec. 18, which claims the benefit under 35 U.S.C. § 119(e) of U.S. Provisional Patent Application 63/612,817, filed 2023 Dec. 20, both of which are incorporated herein by reference in their entirety for all purposes.

### FIELD OF TECHNOLOGY

Aspects of the present disclosure relate to a system, method, and device for stabilizing a distal fracture to a long bone, such as, for example, a human femur.

### BACKGROUND

A fracture near the distal end of a long bone, such as a human femur, can be difficult to stabilize, especially in situations in which the fracture is either caused by or is located near a device such as an artificial knee prosthesis that was previously implanted in the patient. In such situations, it may not be possible to position an intramedullary nail, using conventional techniques and devices, far enough distally in the patient's femur unless the prosthesis has an opening or other recess sized to receive the distal end of the nail. Even still, in patients in which the prosthesis has such an opening, it may still be difficult to position the intramedullary nail, relative to the opening of the prosthesis, in the orientation desired for the fracture to set properly.

As such, bone plates are typically used instead of nails to stabilize distal end fractures in patients with prostheses. However, because bone plates typically are secured to a patient's bone using screws, it can be difficult to secure a plate to the bone of a patient that has osteoporosis because there might not be enough healthy bone to receive the screws securely. Further, using either an intramedullary nail or a bone plate alone in patients with such periprosthetic fractures may not, by itself, provide sufficient “grip” on the broken off distal end of the bone (due to the presence of the prosthetic). In such conditions, the patient likely has an increased risk of a secondary fracture. Similarly, it is difficult to use either an intramedullary nail or a plate, by itself, in patients whose distal femur fragments are small.

### SUMMARY

In some aspects, a system is configured for stabilizing a bone having a distal end fracture and a distal fragment formed by the distal end fracture and a distal end of the bone. The system includes an intramedullary nail, a plate, and a locking fastener. The intramedullary nail is configured to be coupled to the bone such that the intramedullary nail spans the distal end fracture between the bone and the distal fragment. The plate is configured to engage the distal fragment. The locking fastener is configured to couple the intramedullary nail and the plate in a first configuration in which the plate can rotate relative to the intramedullary nail and a second configuration in which the intramedullary nail and the plate are fixedly coupled together.

Other aspects, features, and advantages will be apparent from the following description, drawings, and claims.

### BRIEF DESCRIPTION OF THE DRAWINGS

FIGS. 1A and 1B are side views of schematic representations of a bone stabilization system deployed in a patient in accordance with an aspect of the present disclosure.

FIG. 2 is a front view of the schematic representation of the bone stabilization system of FIG. 1A, deployed in a patient.

FIG. 3 is another front view of the schematic representation of the bone stabilization system of FIG. 1A.

FIG. 4 is a detail view of the schematic representation of a plate of the bone stabilization system of FIG. 1A.

FIG. 5 is a side view of a schematic representation of a bone stabilization system deployed in a patient in accordance with an aspect of the present disclosure.

FIG. 6 is a front view of the schematic representation of the bone stabilization system of FIG. 5 deployed in a patient.

FIG. 7 is a side view of a schematic representation of a bone stabilization system of FIG. 5 deployed in a lateral side of a bone of the patient in accordance with an aspect of the present disclosure.

FIG. 8 is a side view of a schematic representation of a bone stabilization system of FIG. 5 deployed on the medial side of a bone of the patient in accordance with an aspect of the present disclosure.

FIG. 9 is a front view of a bone stabilization system in accordance with another aspect of the present disclosure.

FIG. 10 is a side view of the bone stabilization system of FIG. 9.

FIG. 11 is a top view of the bone stabilization system of FIG. 9.

FIG. 12 is a rear view of the bone stabilization system of FIG. 9.

FIG. 13 is a side view of an intramedullary nail of the bone stabilization system of FIG. 9.

FIG. 14 front view of the intermedullary nail of the bone stabilization system of FIG. 9.

FIG. 15 is another side view of the bone stabilization system of FIG. 9.

FIG. 16 is a perspective view of the bone stabilization system of FIG. 9.

FIG. 17 is another perspective view of the bone stabilization system of FIG. 9.

FIG. 18 is another perspective view of the bone stabilization system of FIG. 9.

FIG. 19A-19D illustrate a method of deploying the bone stabilization system of FIG. 1A, FIG. 5, or 9 in the bone of a patient in accordance with aspects of the present disclosure.

Corresponding reference characters indicate corresponding parts throughout the drawings.

### DETAILED DESCRIPTION

As used herein, the terms “proximal” and “distal” represent relative locations of components, parts, and the like of a bone stabilization system when the bone stabilization system is deployed in a patient's body. For example, a “proximal” component is disposed most adjacent to the patient's torso, a “distal” component is disposed most distant from the patient's torso, and an “intermediate” component is disposed generally anywhere between the proximal and distal components.

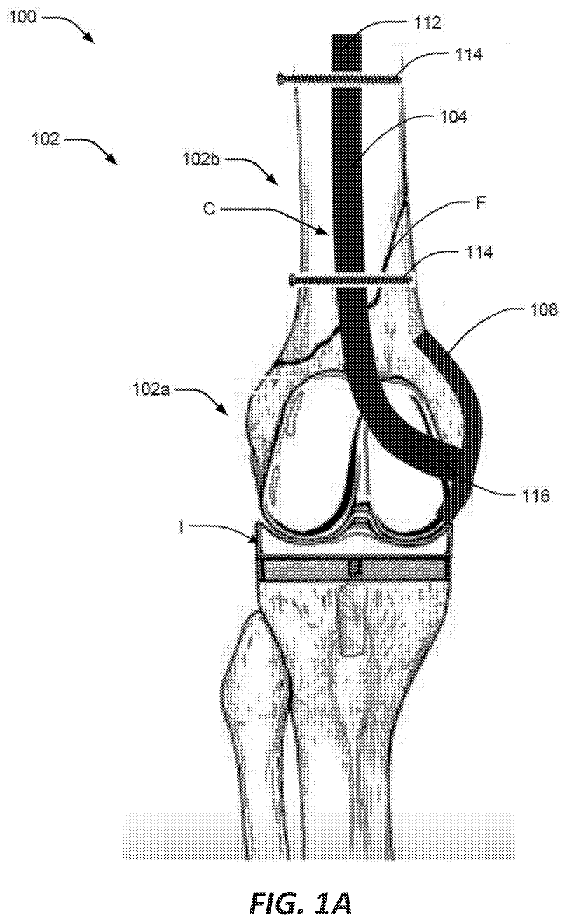

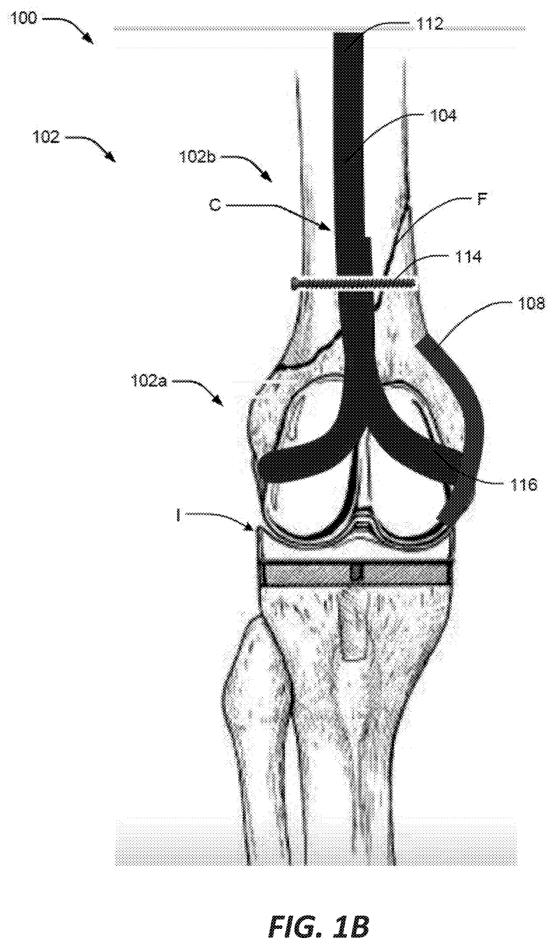

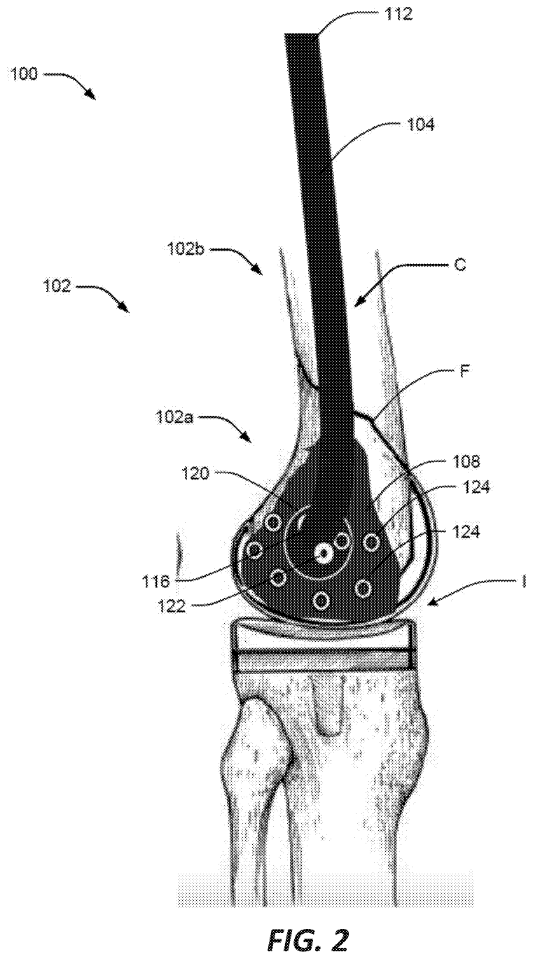

As shown in FIGS. 1-4, a bone stabilization system 100 includes an intramedullary nail 104 and a plate 108. In FIGS. 1-2, the bone stabilization system 100 is shown coupled to a long bone of a patient, for example, a femur 102. In the illustrated aspect, the bone stabilization system 100 is deployed in a patient having a distal end femur fracture F. In such aspects, the fracture F defines a distal femur fragment 102a that may be partially or entirely separated from the remainder of the femur 102b. In some aspects, the patient may have an implant I, such as a prosthetic knee, proximate to the fracture F. In such aspects, the fracture F is a periprosthetic fracture. Periprosthetic fractures can be difficult to stabilize using conventional methods and devices because the implant may prevent or complicate the positioning of an intramedullary nail through the bottom of the distal end of the femur 102. For example, the implant may reduce the amount of the distal end fragment 102a that can be coupled to the intramedullary nail 104.

As shown in FIGS. 1 and 2, the intramedullary nail 104 is configured to be positioned within a medullary cavity C of the patient's femur 102. The intramedullary nail 104 has a first or proximal end 112 and a second or distal end 116. In some aspects, the intramedullary nail 104 is substantially J-shaped. In such aspects, the short end of the J-shape may be positioned proximal to the distal end of the femur 102 (e.g., in the distal femur fragment 102a). In some aspects, the intermedullary nail 104 may be substantially T-shaped. In some aspects, the intermedullary nail may be substantially Y-shaped, as shown, for example, in FIG. 1B. In some aspects, the intramedullary nail 104 may be selected from intramedullary nails having a thickness or diameter that is substantially similar to the diameter of the medullary canal C of the patient.

As shown in FIGS. 1 and 2, the intramedullary nail 104 can be inserted into the femur 102 from the side of the femur 102. The intramedullary nail 104 is positioned in the femur 102 such that the intramedullary nail 104 spans the fracture F. In the illustrated aspect, the intramedullary nail 104 has been inserted through the medial condyle of the femur 102. In other aspects, the intramedullary nail 104 can be inserted through the lateral condyle of the femur 102. The intramedullary nail 104 may be secured within the femur 102 via one or more screws 114. Since the intramedullary nail 104 can be positioned in the femur 102 via the side of the femur 102, the intramedullary nail 104 can be used for fracture(s) F that are near the implant I.

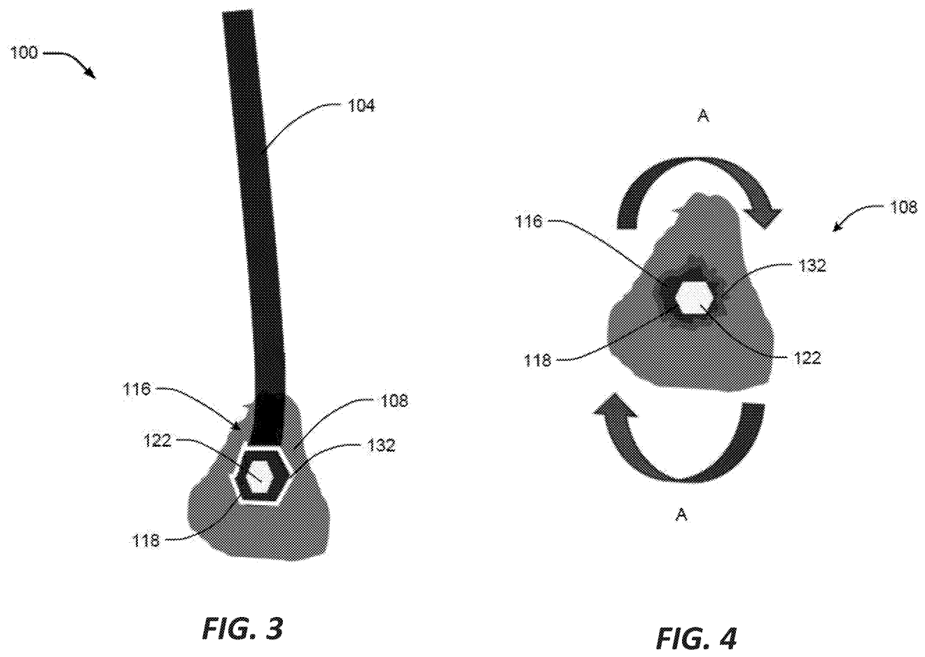

In some aspects, the second end 116 of the intramedullary nail 104 includes one or more locking features 118 (FIG. 3) configured to engage corresponding locking features on the plate 108 and/or a fastener 122, as described in greater detail below. In some aspects, the second end 116 may extend outside of the femur 102.

As shown in FIG. 2, the plate 108 includes an opening 120 and a plurality of screw holes 124. The plate 108 is configured to be coupled to the distal end of the femur 102 by one or more screws 128. In some aspects, the shape or profile of the plate 108 is similar to the shape or profile of the femur 102. In the configuration shown in FIG. 2, the plate 108 is coupled to the distal femur fragment 102a.

The opening 120 is configured to receive the second end 116 of the intramedullary nail 104 or a fastener 122 coupled to the second end 116 of the intramedullary nail 104. In some aspects, the opening 120 is positioned at or near the center of the plate 108. In some aspects, the opening 120 includes locking features 132 configured to engage the corresponding locking features 118 on the intramedullary nail 104 and/or the fastener 122. For example, in some aspects, the locking features 118, 132 may include a plurality of teeth that allow the plate 108 to rotate relative to the intramedullary nail 104 before the plate 108 is locked (or otherwise fixedly coupled) to the intramedullary nail 104. In another example, in some aspects, the locking features 132 may include threads configured to lockingly engage with a head of the fastener 122. For example, in some aspects, the fastener 122 may be a locking screw. In such aspects, a head of the fastener 122 may be configured to lock into threads (e.g., the locking features 132) of the plate 108, and the tail of the fastener 122 may be configured to lock into the locking features 118 of the intramedullary nail 104. In some aspects, the fastener 122 and/or the locking features 118, 132 may be or include a cam lock, a ratchet sleeve nut, a Lovejoy coupling, a jaw flex coupling, internal and/or external splines, a bolt having locking threads at either end and configured to lock to the locking features 118, 132, or combinations thereof.

In some aspects, the plate 108 may be configured to rotate up to 360° relative to the intramedullary nail 104 before the plate 108 is locked to the intramedullary nail 104, as indicated by the arrows A (FIG. 4). The rotation of the plate 108 relative to the intramedullary nail 104 allows the relative orientation of the intramedullary nail 104 and the plate 108 to be optimized based on the location of the fracture F and the shape of the patient's femur 102. In some aspects, the fastener 122 may lock (or otherwise fixedly couple) the intramedullary nail 104 and the plate 108 after the plate 108 has been positioned relative to the intramedullary nail 104 as desired. For example, the plate 108 can be rotated with respect to the intramedullary nail 104 with respect to flexion and extension, such that it works with the implant, and then the plate 108 can be locked in place relative to the intramedullary nail 104.

The use of the intramedullary nail 104 fixedly coupled to the plate 108 is more biomechanically stable than either the intramedullary nail 104 alone or the plate 108 alone. For example, the intramedullary nail 104 traverses down the center of the femur 102, bridging the fracture F, and advances into the distal femur fragment 102a. The use of the intramedullary nail 104 together with the plate 108 can reduce any bending forces in the plate 108 relative to applications in which the plate 108 is used alone. Further, the plate 108 anchors the intramedullary nail 104 in the distal femur fragment 102a, helping to hold the intramedullary nail 104 in place relative to the femur 102 and reducing the risk of secondary fractures. This is particularly advantageous in patients having knee implants I, as the presence of the implant I can reduce the amount of penetration of the intramedullary nail 104 into the distal femur fragment 102a, so locking (or fixedly coupling) the plate 108 to the intramedullary nail 104 helps stabilize and/or anchor the intramedullary nail 104 into the distal femur fragment 102a. In some aspects, once the plate 108 has been locked to the intramedullary nail 104, the bone stabilization system 100 can support the weight of the patient such that the patient can walk or stand. This is advantageous for elderly patients, whose bones typically heal slowly and sometimes do not heal, because such patients can again be ambulatory after deployment of the bone stabilization system 100. In some aspects, it is advantageous to use the bone stabilization system 100 in circumstances in which the smaller portion of the fractured femur 102 is too small to be secured to the larger part of the femur 102b with either an intramedullary nail alone or a plate alone, even in patients not having implants proximate the fracture F.

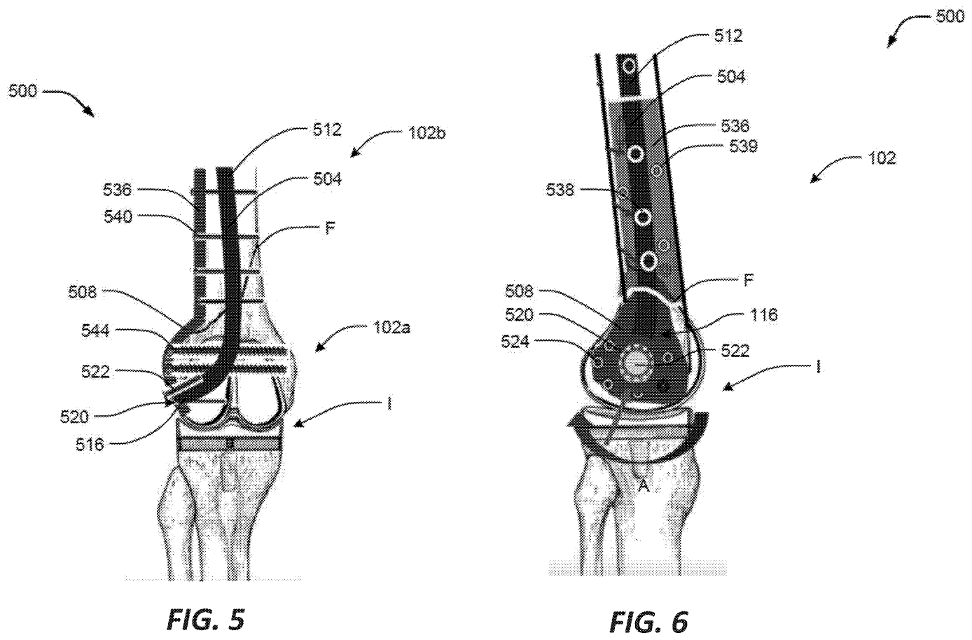

In some aspects, additional intramedullary nails or plates may be used in conjunction with the intramedullary nail 104 and/or the plate 108 described above to provide further stability to the fracture F. For example, in some aspects, additional plates can be coupled to the femur 102 proximate the plate 108 to provide additional support, and/or to better conform to the shape of the femur 102. In such aspects, one or more additional plates can extend to a proximal end of the femur 102. In some aspects, one or more additional plates can be coupled to a lateral side of the femur 102, as shown in FIG. 5. In some aspects, the one or more additional plates can be coupled to a medial side of the femur 102. In some aspects, one or more additional plates can be coupled to both the lateral and medial sides of the femur 102.

In some aspects, the outer surface of the plate 108 may include one or more indentations or grooves. The grooves may be configured to receive a cable or wire therein. In such aspects, the cable or wire is wrapped at least once about a width of the distal femur end, including the plate 108, which may provide more stability to the fracture. In other aspects, the outer surface of the plate 108 may include one or more lips or ears configured to receive the cable or wire therein.

FIGS. 5-8 illustrate further aspects of another example bone stabilization system 500 according to aspects of the present disclosure. Like parts between the bone stabilization system 100 of FIGS. 1-4 and the bone stabilization system 500 of FIGS. 5-8 are described using similar numbers. Corresponding aspects of the bone stabilization system 500 to those already described for the bone stabilization system 100 are omitted for brevity.

The configuration illustrated in FIGS. 5-8 includes one or more second plates 536. The second plate(s) 536 are positioned adjacent to the plate 508. In some aspects, the second plate(s) 536 may be positioned proximal to the plate 508. The second plate(s) 536 may provide additional stability to the fracture F.

As shown in FIG. 6, the second plates(s) include one or more first holes 538 and/or one or more second holes 539. The one or more first holes 538 are configured to receive fasteners 540. In some aspects, one or more fasteners 540 may be coupled between the second plate(s) 536, the intramedullary nail 504, and the femur 102. In some aspects, the first holes 538 may be aligned with corresponding holes in the intramedullary nail 504. In some aspects, the fasteners 540 may be locking screws configured to fixedly secure the second plate 536, the intramedullary nail 504, and the femur 102. The one or more second holes 539 are configured to receive fasteners (not shown). The fasteners are configured to couple the plate 508 to the femur 102 via the second holes 539. In some aspects, the second holes 539 may be arranged in a zig-zag pattern along the plate 508, as shown, for example, in FIG. 6. The fasteners engaged with the second holes 539 may stabilize the plate 508 without going through the intramedullary nail 504.

As shown in FIGS. 5-8, the first plate 508 and the second plate(s) 536 may be coupled to the femur 102 such that the first plate 508 and/or one or more of the second plate(s) 536 span the length of the fracture F. The second plate(s) 536 may also extend along the lateral or medial side of the femur 102 to provide additional support based on the pattern of the fracture F.

In some aspects, after the plate 508 has been locked relative to the intramedullary nail 504, one or more fasteners 544 may be coupled between the plate 508 and the intramedullary nail 504 or the femur 102 near the intramedullary nail 504 to provide more support. In such aspects, the intramedullary nail 504 may include one or more slots configured to receive the fastener(s) 544. In some aspects, the fastener(s) 544 may be positioned substantially oblique to the plate 508.

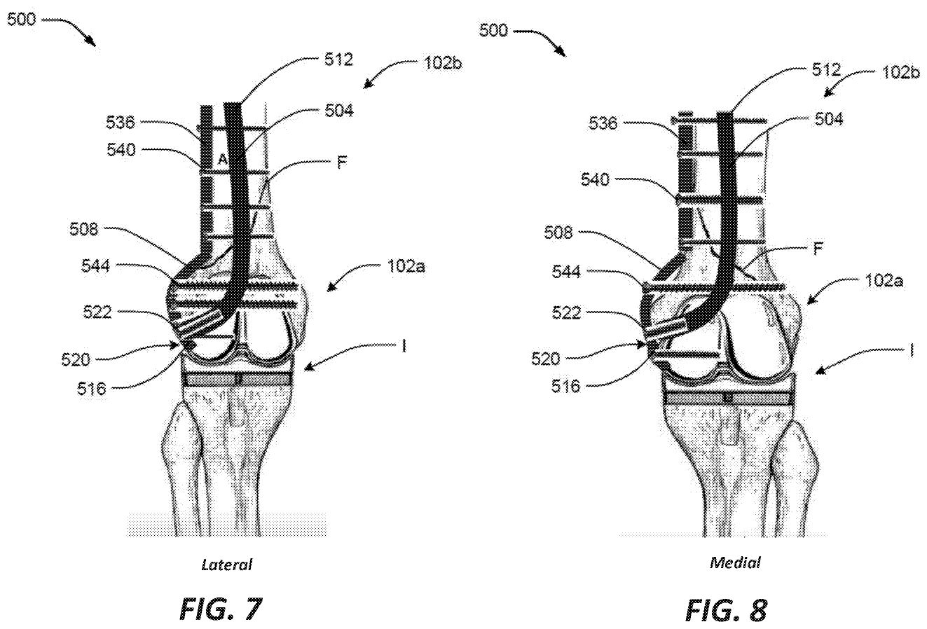

FIG. 7 illustrates the bone stabilization system 500 coupled to the lateral side of a patient's femur 102. FIG. 8 illustrates the bone stabilization system 500 coupled to the medial side of a patient's femur 102.

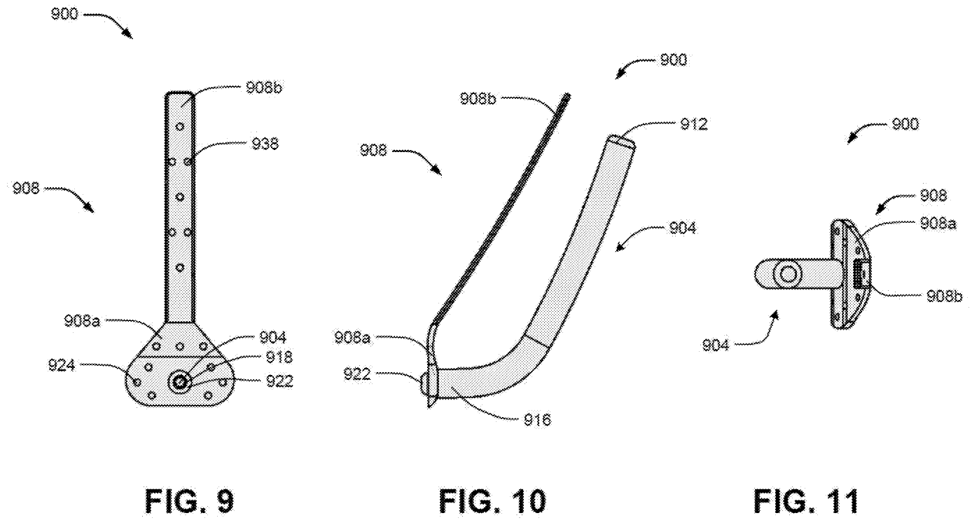

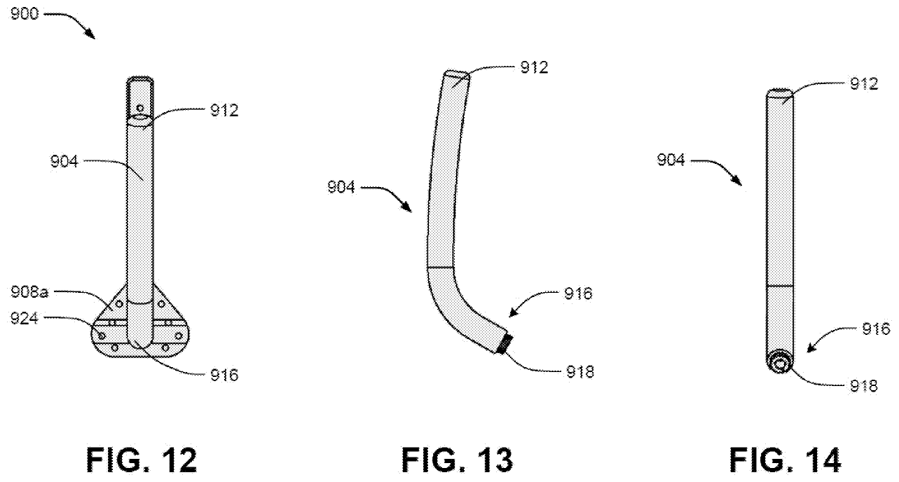

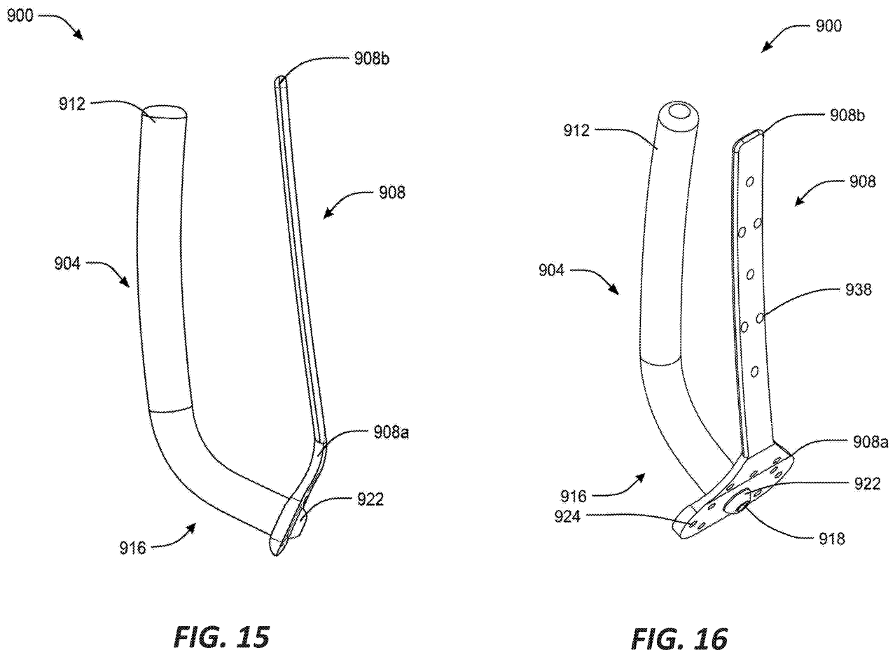

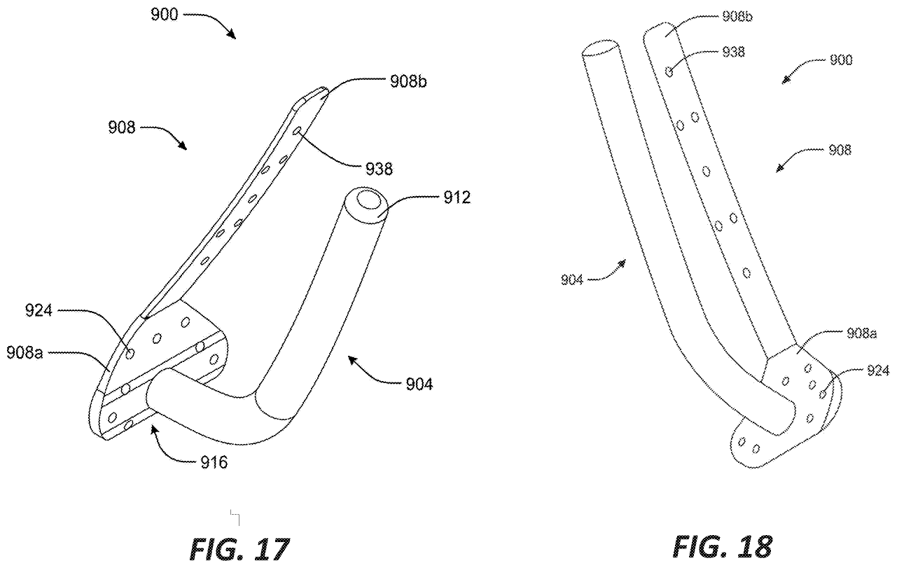

FIGS. 9-18 illustrate further aspects of another example bone stabilization system 900 according to aspects of the present disclosure. Like parts between the bone stabilization system 100 of FIGS. 1-4 and the bone stabilization system 900 of FIGS. 9-18 are described using similar numbers. Corresponding aspects of the bone stabilization system 900 to those already described for the bone stabilization system 100 are omitted for brevity.

In the embodiment illustrated in FIGS. 9-18, the plate 908 includes a first portion 908a and a second portion 908b. The first portion 908a is substantially similar to the plates 108, 508, and is shaped to conform to the distal femur fragment 102a of the femur 102. The second portion 908b is substantially similar to the second plate 536. In the illustrated aspect, the first and second portions 908a,b of the plate 908 are integrally formed. The second portion 908b may be angled relative to the first portion 908a to follow the shape or profile of the femur 102.

FIGS. 13 and 14 illustrate the intramedullary nail 904. In the configuration illustrated in FIGS. 13 and 14, the locking features 918 include a plurality of teeth. The teeth are configured to engage corresponding locking features on the fastener 912 and/or the opening 920 of the plate 908.

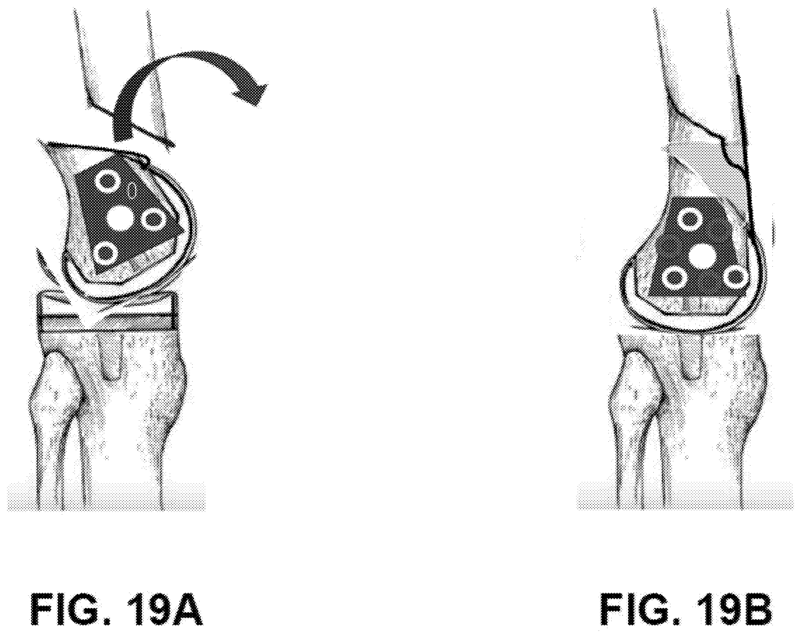

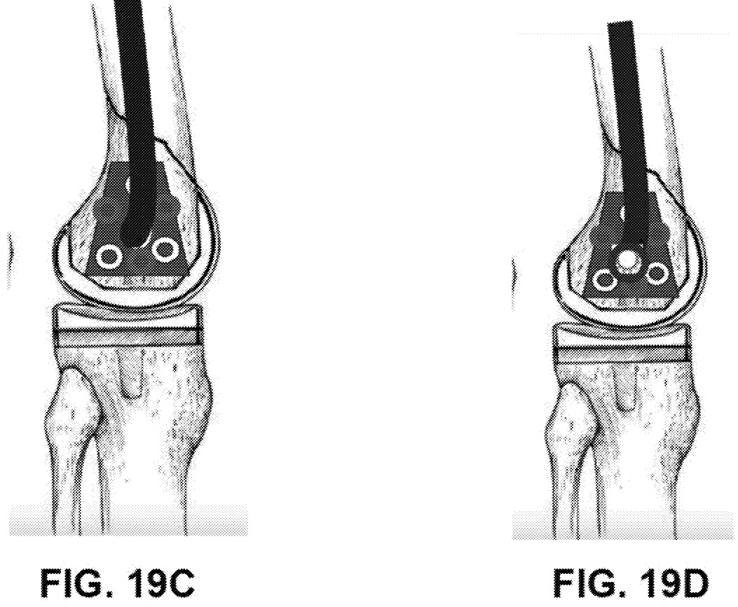

FIG. 19A-19D illustrate a method of deploying the bone stabilization system of FIG. 1A, FIG. 5, or 9 in the bone of a patient in accordance with aspects of the present disclosure.

As shown in FIG. 19A, the plate 108, 508, 908 is coupled to the patient's femur 102. For example, the plate 108, 508, 908 is coupled to a medial or lateral femoral condyle of the distal femur fragment of the patient's femur 102 via the screw holes 124, 524, 924 and the screws 128, 528, 928. The shape of the plate 108, 508, 908 is configured to match the shape and orientation of the patient's medial or femoral condyle. In some aspects in which the plate 908 includes the second portion 908b, only the first portion 908a may be secured to the distal femur fragment at the step illustrated in FIG. 19A.

As shown in FIG. 19B, after the plate 108, 508, 908 has been attached to the distal femur fragment 102a, the patient's femur 102, a tool (not shown) is coupled to the plate 108, 508, 908, for example via holes. The fracture is then spanned and reduced using the tool attached to the plate 108, 508, 908 until the distal femur fragment 102a is aligned or substantially aligned with the remainder of the femur 102b.

As shown in FIG. 19C, the distal femur fragment 102a and the proximal femoral fragment are substantially aligned, and the fracture F is substantially reduced. While the portions of the fractured femur 102 are held in place (e.g., by the tool), a hole is drilled into the cortex of the distal femur fragment 102a proximate the lateral or medial femoral condyle (e.g., at a side of the femur 102). The intramedullary nail 104, 504, 904 is then introduced into the distal femur fragment 102a and advanced into the intramedullary canal C of both the distal femur fragment 102a and larger portion of the femur 102b. The plate 108, 508, 908 can then be rotated as necessary relative to the intramedullary nail 104, 504, 904 as needed to reduce the fracture and align the distal femur fragment 102a with the rest of the femur 102b.

As shown in FIG. 19D, after the intramedullary nail 104, 504, 904 and the plate 108, 508, 908 have been coupled to and/or positioned in the patient's femur 102, the fastener 122, 522, 922 is coupled to the intramedullary nail 104, 504, 904 and the plate 108, 508, 908 (e.g., the first portion 980a of the plate 908), and partially tightened to hold the intramedullary nail 104, 504, 904 and the plate 108, 508, 908 into position.

Reduction of the fracture F is then determined using an imaging system. In some aspects, the imaging system may include C-ram guidance. After reduction of the fracture, F has been confirmed using the imaging system, the fastener 122, 522, 922 is actuated to lock the intramedullary nail 104, 504, 904 to the plate 108, 508, 908 to rigidly fix the intramedullary nail 104, 504, 904 to the plate 108, 508, 908. In aspects that include one or more second plates 536, the second plate(s) 536 may be secured to the femur 102 after the intramedullary nail 504 has been rigidly locked to the plate 508. In aspects in which the plate 908 includes a first portion 908a and the long second portion 908b, the second portion 908b is secured to the femur 102 after the intramedullary nail 904 has been fixedly locked to the first portion 908a of the plate 908. Any additional fasteners, such as, for example, the fasteners 540, 544, are then deployed at this time.

In some aspects, the intramedullary nail 104, 504, 904, and/or the plate 108, 508, 908 may be made of a material that can change shape and/or modulus of elasticity. For example, the material may be able to be changed upon the application of external stimuli such as heat, electricity, and so forth. In some aspects, the material may be nitinol. The shape of nitinol can be changed by the application of a certain temperature or range of temperatures.

In some aspects, the bone stabilization systems 100, 500, 900 may be provided as a kit including various sizes of intramedullary nails 104, 504, 904, various sizes of plates 108, 508, 908, and the fastener 122, 522, 922.

Although the bone stabilization systems 100, 500, 900 are described relative to distal femur fractures, the bone stabilization systems 100, 500, 900 can be used to stabilize fractures on other bones. The bone stabilization systems 100, 500, 900 can be used to treat transitional areas of long bones in which a diaphyseal portion of the long bone transitions into a metaphyseal portion of the long bone with or without a nearby implant. For example, the bone stabilization systems 100, 500, 900 can be used to treat fractures at the distal and/or proximal ends of the tibia. In another example, the bone stabilization systems 100, 500, 900 can be used to treat fractures at the distal end of the humerus, distal or proximal tibia fractures, distal end humerus fractures, and/or distal end radius fractures. In such aspects, the shape or profile of the plates 108, 508, 908 would need to be modified to conform to the shape or profile of the humerus or tibia.

Although the bone stabilization systems 100, 500, 900 described herein are described in the context of periprosthetic fractures, the bone stabilization system 100, 500, 900 can also be used to treat distal end femur fractures, distal or proximal tibia fractures, distal end humerus fractures, and/or distal end radius fractures that occur in patients who do not have prosthetic implants near the fracture site.

It will be appreciated that various implementations of the above-disclosed and other features and functions, or alternatives or varieties thereof, can be desirably combined into many other different systems or applications. Also, that various presently unforeseen or unanticipated alternatives, modifications, variations, or improvements therein can be subsequently made by those skilled in the art, which are also intended to be encompassed by the following claims.

A number of aspects have been described. Nevertheless, it will be understood that various modifications can be made without departing from the spirit and scope of the disclosure. Accordingly, other aspects are within the scope of the following claims.

1. A method for treating a femur having a distal end femur fracture, defining a distal femur fragment and a proximal femoral fragment, the method comprising:
coupling a plate to a medial femoral condyle or a lateral femoral condyle of the distal femur fragment;
positioning the distal femur fragment relative to the proximal femoral fragment to reduce the distal end femur fracture;
introducing an intramedullary nail through a hole in a cortex of the distal femur fragment proximate the lateral femoral condyle or the medial femoral condyle at a side of the femur and advancing the intramedullary nail into an intramedullary canal of both the distal femur fragment and the proximal femoral fragment such that the intramedullary nail spans the distal end femur fracture;
coupling a locking fastener to the intramedullary nail and the plate, the locking fastener being in a first configuration in which the plate is rotatable relative to the intramedullary nail;
rotating the plate relative to the intramedullary nail to reduce the distal end femur fracture and align the distal femur fragment with the proximal femoral fragment; and
actuating the locking fastener into a second configuration to lock the intramedullary nail to the plate and rigidly fix the intramedullary nail to the plate.

2. The method of claim 1, wherein coupling the plate to the medial femoral condyle or the lateral femoral condyle comprises coupling the plate to the distal femur fragment via screw holes in the plate and screws received in the screw holes.

3. The method of claim 1, wherein the plate has a shape conforming to a shape and orientation of the medial femoral condyle or the lateral femoral condyle.

4. The method of claim 1, wherein:
the plate comprises a first portion and a second portion, and
only the first portion is secured to the distal femur fragment before the intramedullary nail is introduced into the distal femur fragment.

5. The method of claim 1, further comprising coupling a tool to the plate after the plate has been attached to the distal femur fragment, wherein positioning the distal femur fragment relative to the proximal femoral fragment comprises spanning and reducing the distal end femur fracture using the tool coupled to the plate.

6. The method of claim 1, wherein the distal femur fragment and the proximal femoral fragment are held in place while the hole is formed in the cortex of the distal femur fragment.

7. The method of claim 1, wherein the hole is formed proximate the lateral femoral condyle.

8. The method of claim 1, wherein the hole is formed proximate the medial femoral condyle.

9. The method of claim 1, wherein the intramedullary nail is positioned within a medullar cavity of the femur and includes a proximal end and a distal end.

10. The method of claim 1, wherein the intramedullary nail is substantially J-shaped.

11. The method of claim 10, wherein a short end of the substantially J-shaped intramedullary nail is positioned proximate a distal end of the femur in the distal femur fragment.

12. The method of claim 1, wherein the intramedullary nail is substantially T-shaped.

13. The method of claim 1, wherein the intramedullary nail is substantially Y-shaped.

14. The method of claim 1, wherein the intramedullary nail has a thickness or diameter substantially similar to a diameter of the intramedullary canal.

15. The method of claim 1, wherein the plate comprises an opening configured to receive a distal end of the intramedullary nail or the locking fastener coupled to the distal end of the intramedullary nail.

16. The method of claim 1, wherein the intramedullary nail comprises one or more locking features configured to engage corresponding locking features on the plate or the locking fastener.

17. The method of claim 16, wherein the locking features include a plurality of teeth that allow the plate to rotate relative to the intramedullary nail before the plate is locked to the intramedullary nail.

18. The method of claim 16, wherein the locking features comprise threads configured to lockingly engage with a head of the locking fastener.

19. The method of claim 16, wherein the locking fastener or the locking features include at least one of a cam lock, a ratchet sleeve nut, a Lovejoy coupling, a jaw flex coupling, internal splines, external splines, or a bolt having locking threads at both ends.

20. The method of claim 1, wherein the plate is rotatable up to 360 degrees relative to the intramedullary nail before the plate is locked to the intramedullary nail.
